(19) United States
(12) Patent Application Publication
(10) Pub. No.: US 2025/0316390 A1
(43) Pub. Date: Oct. 9, 2025

(54) HEALTH DATA ENRICHMENT FOR IMPROVED MEDICAL DIAGNOSTICS

(71) Applicant: Symptoma GmbH, Wien (AT)

(72) Inventors: Jama NATEQI, Lahoff (AT); Thomas LUTZ, Basel (CH)

(21) Appl. No.: 18/864,690

(22) PCT Filed: May 9, 2023
(86) PCT No.: PCT/EP2023/062197

§ 371 Date: Nov. 11, 2024

(30) Foreign Application Priority Data
May 11, 2022 (EP) 22172674.8

(51) Int. Cl. G16H 50/30; G16H 10/60

(52) U.S. Cl. CPC G16H 50/30; G16H 10/60

(57) ABSTRACT

The present invention concerns a computer-implemented method (300) for enriching ambiguous, incomplete or sparse health data, comprising: obtaining an input dataset (302) comprising a plurality of electronic health records (EHRs) associated with a patient; extracting (308) health information which is explicitly recited in the EHRs from the input dataset (302), including at least one diagnosis indicated by a name of a disease (504) or a medical classification code (502) which denotes a disease (504); generating (310) supplementary health information which is not explicitly documented in the EHRs based, at least in part, on the extracted health information, the supplementary health information including at least one or more symptoms (506) inferred from diseases (504) directly or indirectly documented in the input dataset (302); and validity-scoring (312) at least part of the extracted health information and the supplementary health information to produce an output dataset (314).

### TECHNICAL FIELD

The present invention generally relates to the field of computer-aided health management, and more specifically to techniques for enriching otherwise ambiguous, incomplete, or incorrect health data to enable improved medical diagnostics.

### BACKGROUND

In today's healthcare market, around one in seven diagnoses are misdiagnoses, which accounts for EUR 446 billion in wasted costs in the EU and USD 750 billion in the US. Solving misdiagnosis could free up 30% of total healthcare budgets globally, but a solution is not in sight. This is in part because of the existence of more than 20,000 diseases, which cognitively overwhelm even the world's best doctors and make today's healthcare largely depend on subjective decision-making.

The problem of misdiagnoses is even more severe in the context of rare diseases. In the EU, a disease is commonly defined as rare when it affects fewer than 1 in 2,000 people. In the US, a rare disease is defined in the Orphan Drug Act of 1983 as a condition that affects fewer than 200,000 people in the US. According to some statistics, to this day 22 of 30 million patients with rare diseases in the EU do not have a diagnosis in the first place, and eight million patients with a diagnosis had have to wait for 10 years on average to get it. Each year, 1.5 million lives could be saved globally with the right diagnosis.

Furthermore, pharma companies with drugs for rare diseases lose EUR 412 billion in revenue potential globally due to misdiagnosis alone. As an example, one of the leading companies specializing in drugs for rare diseases (orphan drugs) has invested EUR 2.25 million in educational campaigns targeted at a German doctoral community only to find one single patient over a course of three years. This approach is insufficient and yet demonstrates the dimension of market demand for rare disease diagnosis.

Additional complexity is introduced by the way hospitals and other healthcare institutions typically collect and manage the data relating to their patients. Typically, each hospital has a unique combination of software to store and handle electronic health records (EHRs) in hospital information systems (HIS). The data in these systems is often unstructured, e.g., comprising free text such as doctor's notes. Even if there is structured data, e.g., lab values measured by machines, it is often not very well-defined, e.g., not classified by international standards, such as Logical Observation Identifiers Names and Codes (LOINC). In total, the data is typically ambiguous, incomplete, and too often even wrong.

In the meantime, in the area of computer-aided diagnosis, approaches have been proposed to improve the accuracy of medical diagnosis with the help of machine-learning techniques.

For example, U.S. U.S. Pat. No. 11,017,905 B2 of Babylon Partners Limited titled “Counterfactual measure for medical diagnosis” discloses a computer-implemented medical diagnosis method which includes receiving an input from a user comprising at least one symptom, and providing the at least one symptom as an input to a medical model. The medical model includes a probabilistic graphical model comprising probability distributions and relationships between symptoms and diseases. The method also includes performing inference on the probabilistic graphical model to obtain a prediction of the probability that the user has that disease. The method also includes outputting an indication that the user has a disease from the Bayesian inference, wherein the inference is performed using a counterfactual measure. Further background information can be found in Richens, J. G., Lee, C. M. & Johri, S. “Improving the accuracy of medical diagnosis with causal machine learning”. Nat Commun 11, 3923 (2020). https://doi.org/10.1038/s41467-020-17419-7.

International Application No. WO 03/040965 discloses a data mining framework for mining high-quality structured clinical information. The data mining framework includes a data miner that mines medical information from a computerized patient record (CPR) based on domain-specific knowledge contained in a knowledge base. The data miner includes components for extracting information from the CPR, combining all available evidence in a principled fashion over time, and drawing inferences from this combination process. The mined medical information is stored in a structured CPR which can be a data warehouse.

International Application No. WO 2016094450 discloses a rare disease matching and prediction portal where a number of symptoms are matched with a list of rare diseases to produce candidate diseases, after which the candidate diseases are evaluated based on weighted lists of symptoms for each candidate disease to produce a confidence indicating a likelihood that a patient suffers from one of the rare diseases. Information curated from publications and other curated databases related to rare diseases are utilized to determine the weighted list of symptoms for each disease based on prevalence relevance of each symptom to each candidate disease, and a customized algorithm is applied to determine the confidences for the candidate diseases. Along with the confidences, the portal may provide a disease profile for each disease which includes possible treatments for the candidate diseases. The patient may then be treated for at least one of the candidate diseases based on the confidences.

U.S. Patent Application Publication No. 2019/189253 discloses a medical condition verification system. The medical condition verification system receives patient electronic medical record (EMR) data and parses the patient EMR data to identify an instance of a medical code or medical condition indicator present in the patient EMR data. The medical condition verification system performs cognitive analysis of the patient EMR data to identify evidential data supportive of the instance referencing an associated medical condition. The medical condition verification system generates a measure of risk of the patient having the medical condition based on the identified evidential data and based on a machine learned relationship of medical factors in patient EMR data relevant to generating the measure of risk for the associated medical condition. The medical condition verification system generates an output representing the measure of the risk of the patient having the associated medical condition.

U.S. Patent Application Publication No. 2021/193320 discloses a system for identifying a probability of a medical condition in a patient. The method includes a processor obtaining data set(s) related to a patient population diagnosed with a medical condition and based on a frequency of features in the data set(s), identifying common features and weighting the common features based on frequency of occurrence in the data set(s) to generate mutual information. The processor generates pattern(s) including a portion of the common features to generate a machine learning algorithm(s). The processor compiles a training set of data to use to tune the machine learning algorithm(s). The processor dynamically adjusts common features in the pattern(s) such that the machine learning algorithm(s) can distinguish patient data indicating the medical condition from patient data not indicating the medical condition. The processor applies the machine learning algorithm(s) to data related to the undiagnosed patient, to determine the probability.

U.S. Patent Application Publication No. 2021/233658 discloses a computer-implemented method for medical diagnosis, comprising: receiving a user input from a user, the user input comprising an input symptom; determining a measure of relevance of a plurality of items of medical data to the user input, wherein the plurality of items of medical data are items of medical data for which information associated with the user is stored; determining whether to include the stored information corresponding to an item of medical data in a first set of information, based on the measure of relevance for the item of medical data; providing the user input and the first set of information as an input to a model, the model being configured to output a probability of the user having a disease; and outputting a diagnosis based on the probability of the user having a disease.

However, the known solutions typically use the available raw data without further interpretation or preprocessing to build machine-learning models. Since the models are thus based to a significant extent on missing and/or wrong data, this has a severe negative impact on their predictive performance.

It is therefore a problem underlying the invention to provide a technique for improving the quality in health information as a foundation for more accurate diagnoses and thereby overcome overcoming the above-mentioned disadvantages of the prior art at least in part.

### SUMMARY OF THE INVENTION

Embodiments of the invention provide technology to unlock objective reasoning in medical decision making. In one embodiment, a computer-implemented health data enrichment method is provided. The method may comprise obtaining an input dataset. The input dataset may comprise health information, preferably in the form of a plurality of electronic health records (EHRs), associated with a patient. The method may comprise extracting health information from the input dataset. The health information may include at least one of a diagnosis, a treatment, a risk factor, a lab value, a sign, a biosignal, an image and a free-text observation. More generally, the health information may comprise any information relevant to the medical status of the patient. For example, in addition to, or alternatively to the examples mentioned above, the health information may comprise any selection from the following:
Physiological parameters (also referred to as vital parameters), such as blood pressure, pulse, heart rate, body temperature, respiratory rate, oxygen saturation, pain score, blood sugar level, age, weight, ethnicity, bodyfat, height, sex, blood type, motion-based indicators (e.g., to identify and/or track Parkinson's Disease symptoms) and/or summarized indicators (e.g., body mass index, Glasgow coma scale)
Non-physiological parameters, such as geography/GPS position, outside temperature, date/season, profession, education, religion and/or marital status
Lifestyle-related parameters, such as physical excretion, physical activity, stress, eating habits, sleeping habits, voice analysis, emotion recognition, social contacts and/or exposure to health risk environments (e.g., radiation exposure, contamination exposure, fine dust pollution and/or noise, e.g., from noise maps)

The sources of such health information may include mobile devices such as smartphones, e.g., by way of various sensors associated with rotation, acceleration, barometer, fingerprint, electromagnetic sensor, brightness sensor, heart rate monitor, proximity sensor, GPS sensor, magnetometer, microphone, image sensor, touch sensor, humidity sensor and/or LIDAR sensor. The sources of such information may also include stationary devices such as smart home devices (e.g., a smart kitchen to derive eating habits or a smart bathroom to analyze sewage) or specialist devices (e.g., laboratory devices, hospital devices or doctor's devices).

The method may comprise generating supplementary health information based, at least in part, on the extracted health information. The supplementary health information may include at least one of a disease and a symptom. The method may comprise validity-scoring at least part of the extracted health information and the supplementary health information to produce an output dataset.

Accordingly, the above aspect of the invention provides a way for health data of a patient, which is typically ambiguous, incomplete, or even incorrect, to be enriched to improve the quality of the health data, effectively restoring the patient's phenotype from sparse data. The disclosed algorithms may be configured for reverse-engineering signs and/or symptoms, including their probability, from documented diagnoses and, optionally, other supporting disease-related data. Health data “enrichment” is to be understood broadly in this context, and the process of enriching may include preprocessing data, validating data, refining data, correcting data, verifying data, falsifying data, assessing data as to its credibility, or any combination thereof.

In another aspect of the method, generating the supplementary health information may comprise determining, using a code-disease mapping, one or more diseases associated with a medical classification code documented in the input dataset. Accordingly, the method may infer possible (candidate) diseases from medical classification codes documented in the input dataset. The person skilled in the art knows several such classification codes, e.g., ICD-10, which is the 10^{th} revision of the International Statistical Classification of Diseases and Related Health Problems (ICD), a medical classification list by the World Health Organization (WHO).

In another aspect, generating the supplementary health information may comprise determining, using a disease-symptom mapping, one or more symptoms associated with a disease documented in the input dataset and/or determined using the above-explained aspect. Accordingly, symptoms may be inferred from the diseases directly or indirectly documented in the input dataset. To this end, the disease-symptom mapping may comprise a database of diseases and likely their symptoms, preferably annotated with probabilities.

In yet another aspect, generating the supplementary health information may comprise determining, using a drug-symptom mapping and/or a drug-disease mapping, one or more symptoms and/or diseases associated with a drug documented in the input dataset. Accordingly, the data may be enriched even more based on drugs and/or other treatments prescribed by the healthcare professional, as documented in the input dataset.

Generating the supplementary health information may be based on an ontology, i.e., a data structure which defines the relevant concepts and their relationships. The ontology may comprise the above-mentioned code-disease mapping, the disease-symptom mapping, the drug-symptom mapping and/or the drug-disease mapping.

Furthermore, the validity-scoring may comprise ranking diseases and/or symptoms based on a credibility associated with a source of the respective disease and/or symptom. Documented lab values, signs and/or biosignals may indicate a highest credibility. Medical classification codes used for a diagnosis may indicate a second highest credibility. Prescribed treatments and/or drugs may indicate a third highest credibility. Symptoms documented in free text may indicate a lowest credibility. This way, the enriched data is further refined in that the inferred parts are validated depending on their context, which further improves the quality of the data.

In one aspect, the validity-scoring may comprise scoring a symptom derived from a lab value or a sign with a first validity factor, wherein the first validity factor is preferably 100%. The validity-scoring may also comprise scoring a symptom derived from a biosignal with a second validity factor, wherein the second validity factor preferably depends on an analysis module associated with the biosignal. Further, scoring a disease derived from a diagnosis or a prescribed treatment with a third validity factor may be provided, wherein the third validity factor is based, at least in part, on one or more risk factors of the patient, if present in the input dataset.

Each of the plurality of EHRs in the input dataset may comprise a timestamp, and the method may further comprise sorting the input dataset by timestamp. Furthermore, the method may comprise clustering the input dataset into one or more clusters based, at least in part, on the timestamps. The step of extracting health information may be performed for each cluster. Accordingly, this aspect results in a particularly contextual data enrichment, since the input EHRS, which may span a considerable timespan, are clustered into time-related and therefore likely also contextually related clusters.

Obtaining the input dataset may comprise exporting the plurality of EHRs from a hospital information system (HIS). The exported plurality of EHRs may comprise all EHRs associated with the patient available in the HIS. Accordingly, a screening process for the EHR data of a given patient in the HIS is provided.

The method may also comprise anonymizing the exported plurality of EHRs, to ensure that no sensitive patient-related data is accessible by non-authorized entities.

The exporting and the anonymizing may be performed by a data processing system which is deployed locally within an IT infrastructure of the hospital comprising the HIS. The data processing system may be configured for communicating with the HIS only via a secured local network connection. Accordingly, such an on-site screening process is particularly secure.

Extracting the health information may comprise processing the input dataset using a feature extraction method for text classification, such as any suitable classification technology known to the person skilled in the art.

The method may comprise outputting the output dataset on a display of an electronic device. The electronic device may be associated with a healthcare professional for use in computer-aided diagnosis, or associated with the patient.

In addition or alternatively, the method may comprise providing the output dataset as an input to a computer system for further use, and/or to a machine-learning model or machine-learning algorithm.

The present invention also provides a computer program comprising instructions which, when the program is executed by a computer, cause the computer to carry out any of the methods disclosed herein.

A data processing system may also be provided comprising means for carrying out any of the methods disclosed herein. The data processing system may be deployed locally within an IT infrastructure of a hospital comprising a hospital information system (HIS). The data processing system may be configured for communicating with the HIS only via a secured local network connection.

### BRIEF DESCRIPTION OF THE DRAWINGS

FIG. 1 shows an example of a health data screening process in which embodiments of the invention may be applied.

FIG. 2 shows an overview of a system in which embodiments of the invention may be practiced.

FIG. 3 shows a data enrichment process in accordance with embodiments of the invention.

FIG. 4 shows an exemplary clustering of EHR data in accordance with embodiments of the invention.

FIG. 5 shows an exemplary ontology which associates medical classification codes, diseases, symptoms and drugs in accordance with embodiments of the invention.

FIG. 6 shows an example of an ICD code and its relations to multiple diseases in accordance with embodiments of the invention.

FIG. 7 shows an exemplary disease and an (incomplete or partially complete) list of associated symptoms with probabilities of presentation in percent in accordance with embodiments of the invention.

FIG. 8 shows an exemplary treatment/drug and a selection of indications (various symptoms and diseases) in accordance with embodiments of the invention.

FIG. 9 shows an example of an enriched health information dataset with direct data and indirect data in accordance with embodiments of the invention.

FIG. 10 shows an exemplary process for validity-scoring enriched health information in accordance with embodiments of the invention.

### DESCRIPTION OF PREFERRED EMBODIMENTS

Embodiments of the invention generally aim at eliminating the guesswork from medicine to ensure that every patient receives the right diagnosis and treatment. In particular, the disclosed techniques aim at helping to diagnose patients with rare or even ultra-rare diseases. Certain embodiments provide improved techniques for collecting, preparing, processing and/or analyzing patient-related health data which originates from electronic health records as they are commonly used in hospital information systems, or other sources. Certain embodiments, enable healthcare providers such as hospitals and doctors to automate their diagnostic pathways, resulting in increased healthcare quality and accuracy, which could eventually save 1.5 million lives of patients yearly by supporting doctors and hospitals in making the right diagnoses.

As used herein, the term “health record” or “medical record” may refer to a systematic documentation of a patient's medical history and care across time, typically within one particular health care provider's jurisdiction. A health record may include a range of health data, including demographics and social information, full medical history including reports by doctors, nurses and discharge letters, medication and allergies, immunization status, laboratory test results, radiology images and reports, vital signs and related measurements, personal statistics and risk factors such as age and weight, and even billing information. A health record typically includes a variety of types of notes entered over time by healthcare professionals, recording observations and administration of drugs and therapies, orders for the administration of drugs and therapies, test results, x-rays, reports, and the like.

As used herein, the term “electronic health record” (EHR) may refer to a health record in a digital format. Several specifications and standards have been developed for storing EHRs in an interchangeable format. One such exemplary standard is Fast Healthcare Interoperability Resources (FHIR) created by the Health Level Seven International (HL7) health-care standards organization which describes data formats and elements (referred to as “resources”) and an application programming interface (API) for exchanging EHRs. Another example is openEHR, which is an open standard specification in health informatics maintained by the openEHR Foundation which describes the management, storage, retrieval and exchange of health data in EHRs.

As used herein, the term “hospital information system” (HIS) may refer to a hardware- and/or software-based information system configured for managing aspects of a hospital's operation, such as the storage and processing of medical information including EHRs, but also other aspects such as administrative, financial and/or legal aspects. Hospital information systems may also be referred to as hospital management software (HMS) or hospital management system.

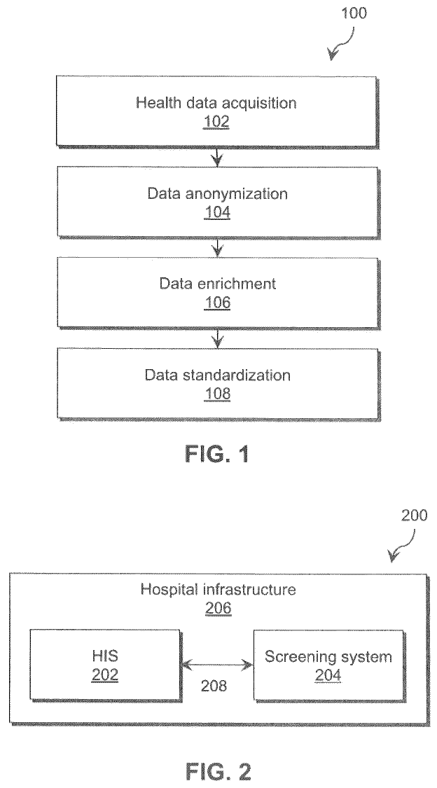

FIG. 1 shows an example of a health data screening and/or verification process 100 in which embodiments of the invention may be practiced. In the illustrated embodiment, the process 100 starts with a health data acquisition step 102. The acquired health data is anonymized in step 104, and the anonymized health data is input into a data enrichment process 106. The data anonymization step 104 may be performed already by the HIS of the hospital. The anonymization may comprise removing any identifiable data, such as names. Furthermore, the anonymization may comprise encrypting patient IDs with a cryptographic key in the possession of the hospital. In step 108, the output of the data enrichment process 106 is mapped to a standardized format for further processing. The person skilled in the art will understand that certain steps of the process 100 may be omitted, e.g., the data anonymization step 104 and/or the data standardization step 108, depending on the circumstances. The resulting output data may be of improved accuracy and quality as compared to the input data, which is typically ambiguous, incomplete, and oftentimes plain wrong. The output of the screening process 100 may be the basis for various further analyses, as otherwise disclosed herein, and as apparent to the person skilled in the art.

In certain embodiments, the data acquisition step 102 comprises exporting health data from EHRs stored in a HIS. The EHR health data may be stored in the HIS in accordance with one of several specifications and standards known in the field, as mentioned above.

In addition or as an alternative, embodiments of the invention may be capable of processing non-digital health data. Such non-digital health data may be present, e.g., in paper-based health records. Irrespective of the carrier (paper-based vs. electronic), the stored information may be substantially the same as in the above-mentioned EHRs. In such scenarios, the data acquisition step 102 may comprise digitizing health records using various techniques. For example, paper-based health records may be scanned using a scanner device and the information may be made digitally accessible by transferring it into machine-encoded text using optical character recognition (OCR) software. As another example, spoken language, e.g., in recordings of dictated doctor's notes, may be converted to machine-encoded text using automatic speech recognition software.

FIG. 2 shows an overview of a system 200 in which embodiments of the invention may be practiced. The system 200 comprises a HIS 202 and a screening system 204. The screening system 204 may be a computer system configured for performing the health data screening process 100, or at least parts thereof. In certain embodiments, at least the data acquisition step 102 and the data anonymization step 104 are performed on-site, i.e., locally with respect to the HIS 202, so that the data never leaves the hospital IT infrastructure 206. This may involve deploying a dedicated computer system 204 locally within the IT infrastructure 206 of the hospital. The screening system 204 may be configured for communicating with the HIS 202 only via a secured local network connection 208.

### Ontology-Based Data Enrichment

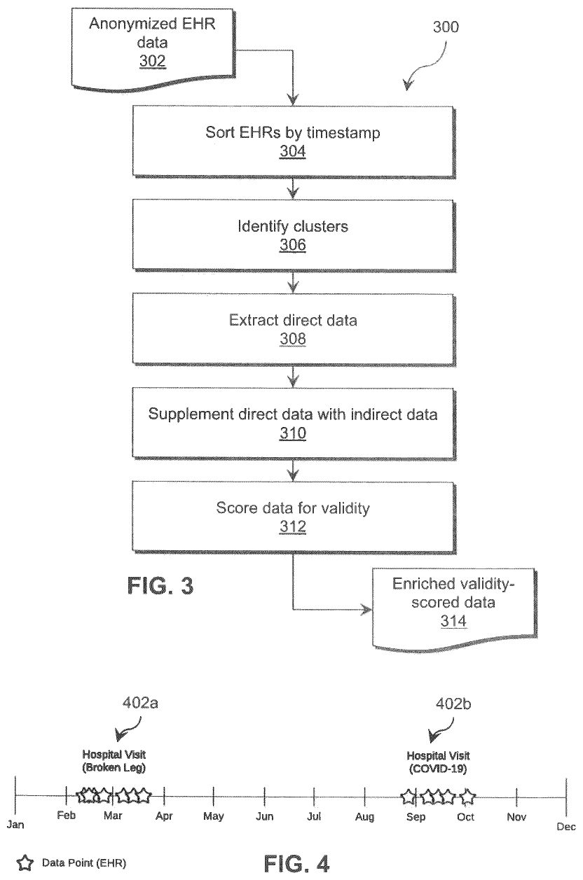

FIG. 3 shows a data enrichment process 300 according to an embodiment of the invention, which may be an example of the data enrichment step 106 in FIG. 1. Generally, the data enrichment process 300 serves for enriching the available data extracted from the HIS 202 by restoring plausible missing data and scoring the data for validity. The input to the data enrichment process 300 comprises an EHR dataset 302. In the illustrated embodiment, the EHR dataset 302 comprises a full set of available (anonymized) EHRs of a single patient, i.e., all EHRs of the patient which are available at the HIS 202. Each EHR in the dataset 302 is associated with a timestamp. The output of the data enrichment process 300 comprises enriched and validity-scored output data 314.

In step 304, the EHRs in the EHR dataset 302 are sorted by timestamp, and clusters 402 are identified in step 306. In one embodiment, the clusters 402 are created based on the timestamps associated with each EHR using a clustering method such as the Jenks natural breaks classification method, for example. Each cluster represents an episode in the patient's life where the symptoms presented in the corresponding EHRs can be assumed to be likely caused by the same underlying conditions. FIG. 4 shows an exemplary clustering of an EHR dataset 302. In the illustrated example, the clustering has resulted in a first cluster 402a of EHRs from a first hospital visit, so that the EHR data in this cluster is likely associated with a broken leg, and a second cluster 402b of EHRs from a second hospital visit, so that the EHR data in this cluster is likely associated with COVID-19. It goes without saying that such a clustering may result in any number of clusters 402, including a single cluster, depending on the information in the EHR dataset 302. Referring back to FIG. 3, after the EHR dataset 302 has been clustered, the processing continues by processing the EHRs in each cluster separately in the illustrated embodiment. Regardless of how many clusters 402 were created, how many EHRs are clustered into a given cluster 402, or whether the EHR dataset 302 has been clustered at all, the process 300 proceeds with extracting health information from the EHR dataset 302.

In step 308, health information is extracted from the EHR dataset 302 (preferably for a given cluster 402, as explained above), namely health information which is explicitly recited in the EHR dataset 302, which health information is also referred to herein as “direct data”. Extracting the direct data may involve suitable feature extraction methods for text classification, which are available to the person skilled in the art.

The direct data extraction step 308 may consider any of the following information, depending on the content of the respective EHR data point:
one or more diagnoses, e.g., the name of a disease or a medical classification code which denotes a disease, such as an ICD code
one or more symptoms, e.g., abnormal lab signs, biosignals such as ECG/EEG, or described in free text, e.g., doctor's notes
one or more treatments, e.g., drugs prescribed by the doctor
one or more risk factors, such as age, sex, ethnicity

The direct data is supplemented with additional health information in step 310, which is also referred to herein as “supplementary health information” or “indirect data”, to create an enriched health information dataset.

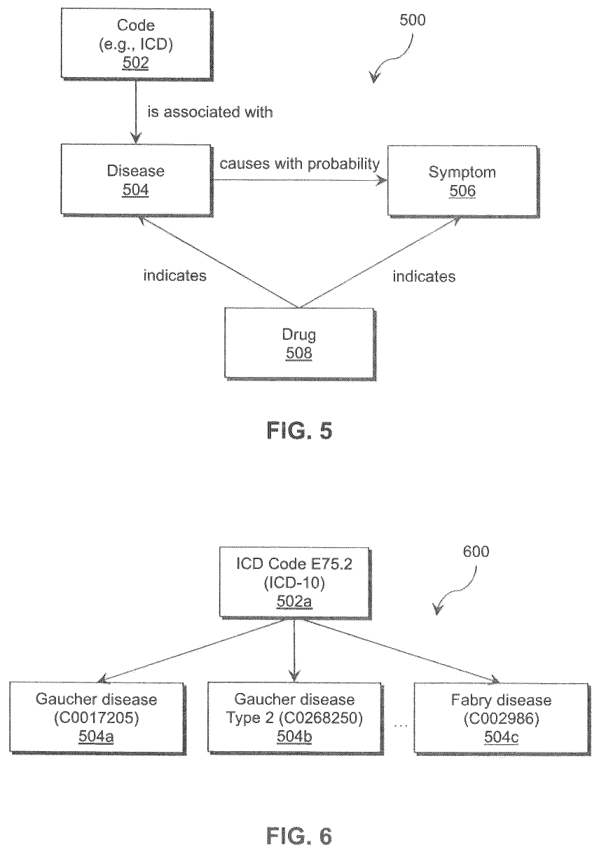

In certain embodiments, the data supplementation 310 is performed based on an ontology 500, an example of which will now be explained with reference to FIG. 5. In the illustrated embodiment, the ontology 500 comprises:
diseases 504 and their relationship with codes 502 of medical coding systems, such as International Classification of Diseases (ICD), i.e., information about which code 502 is associated with which disease(s) 504
drugs 508 and their relationship with diseases 504 and symptoms 506, i.e., which drug 508 indicates which disease(s) 504 and/or which symptom(s) 506 symptoms 506 and their relationship with diseases 504, i.e., the probability that a symptom 506 occurs for a certain disease 504

Each item in the ontology 500 may comprise an identifier (ID) for unique identification. The concepts and relationships of the ontology 500 may be defined in an ontology specification language available to the person skilled in the art, such as the Web Ontology Language (OWL), for example. The data of the ontology 500 may be organized as a database, for example a relational database or any other suitable data organization technique.

Returning to FIG. 3, the data supplementation step 310 comprises in the illustrated embodiment the following (or any subset of the following) steps:

For each diagnosis which is recited in the EHR dataset 302 in the form of a medical classification code, a list of relevant diseases 504 is retrieved via the ontology 500. Note that a diagnosis described via a medical classification code may not necessarily be unambiguous but may represent a multitude of actual diseases. For example, FIG. 6 shows an exemplary code-disease mapping 600 which defines that the ICD-10 code E75.2 is associated with several diseases 504, such as Gaucher disease 504a, Gaucher disease type 2 504b, Fabry disease 504c and others. Instead of querying the ontology 500, it is also conceivable to query a dedicated database which maps medical classification codes to associated diseases, such as, e.g., https://www.icd-code.de/.

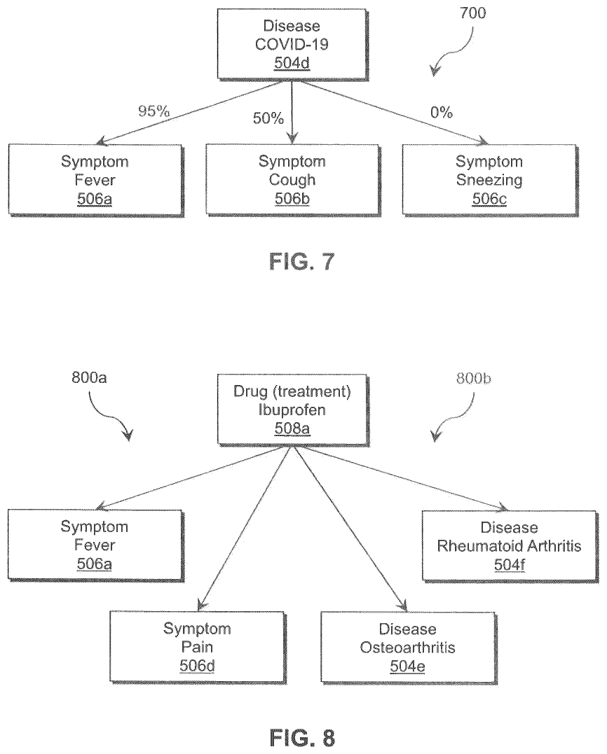

For each disease 504 (i.e., the diseases recited explicitly in the EHR dataset 302 and the diseases derived indirectly, as explained above), one or more associated symptoms 506 are retrieved via the ontology 500. Note that a disease 504 may be associated with multiple symptoms 506. For example, FIG. 7 shows an exemplary disease-symptom mapping 700 which defines that the disease 504d (COVID-19) is associated with symptoms 506a (fever), 506b (cough) and 506c (sneezing). In the illustrated embodiment, each association is additionally qualified by a percentage. Accordingly, the mapping 700 represents the probability of each symptom 506 to be present for the associated disease 504. As one exemplary use, the probability may be used for weighting in subsequent processing, e.g., in machine-learning models.

For each treatment comprising a drug 508 in the EHR dataset 302, one or more associated symptoms 506 and/or diseases 504 are retrieved via the ontology 500. It is assumed that the drugs 508 recited in the EHR dataset 302 were prescribed as indicated or, when prescribed off-label, as indicated via guidelines and/or good medical practice, or commonly prescribed, even when wrongfully. FIG. 8 shows an exemplary drug-symptom mapping 800a and drug-disease mapping 800b which define that the drug 508a (Ibuprofen) is associated with symptoms 506a (fever) and 506d (pain) as well as with diseases 504e (osteoarthritis) and 504f (rheumatoid arthritis). The mappings 800a and 800b may be provided as a combined drug-disease mapping in certain embodiments.

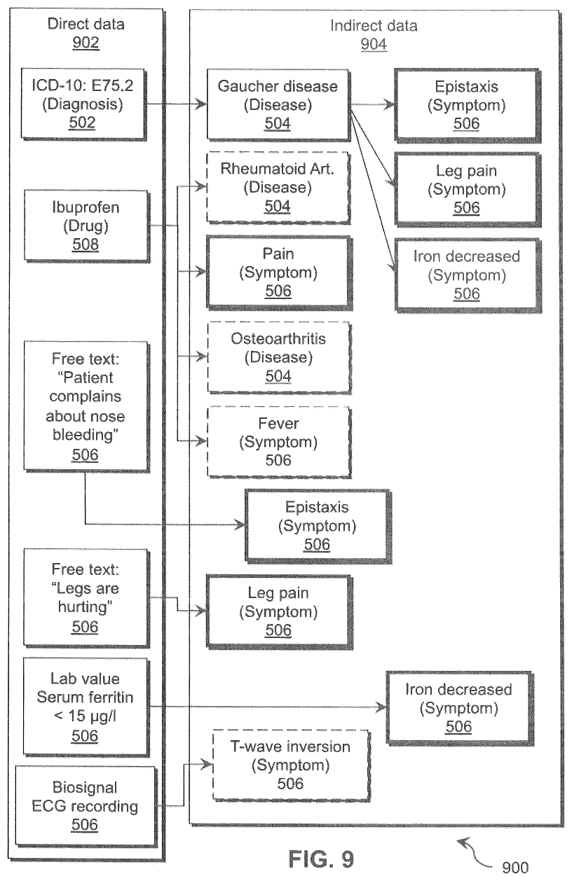

Overall, the result of the data gathering involved in the data extraction step 308 and data supplementation step 310 is an enriched health information dataset, an example of which will now be described in connection with FIG. 9. As can be seen, the enriched health information dataset 900 comprises both direct data 902, i.e., data which was explicitly documented in the EHRs, and indirect data 904, i.e., data which has been generated throughout the processes described herein, and which can be regarded as having been only implicitly documented in the EHRs.

In the illustrated example, the direct data 902 comprises a diagnosis 502 indicated by ICD-10 code E75.2 and a drug 508, in this case Ibuprofen, prescribed by the doctor as a result of the diagnosis. The direct data 902 further comprises two symptoms documented in portions of free text, a first free text noting “Patient complains about nose bleeding” and a second free text noting “Legs are hurting”. The direct data 902 further comprises a symptom 506 documented in the form of a lab value, in the example “serum ferritin <15 μg/l”, and a further symptom 506 in the form of a biosignal, namely an ECG recording.

As illustrated, the indirect data 904 comprises an indication of Gaucher disease 504 derived from the diagnosis 502 in the direct data 902, as well as associated symptoms 506 epistaxis, leg pain and decreased iron. As a result of the Ibuprofen prescription documented in the direct data 902, the indirect data 904 comprises two further diseases 504, namely rheumatoid arthritis and osteoarthritis, as well as two further symptoms 506, namely pain and fever. Based on the free text symptom 506 documenting the observation of a bleeding nose, the symptom epistaxis 506 was derived, and the symptom leg pain 506 based on the corresponding free text symptom 506. The lab value symptom 506 resulted in the symptom “iron decreased” 506. The biosignal symptom 506 caused a symptom 506 “T-wave inversion” in the indirect data 904.

As can be seen, some datapoints (symptoms 506, diseases 504, or the like) may be supported with redundant evidence, which are marked with bold lines in FIG. 9. Other datapoints may have no further evidence and are marked with dashed lines in FIG. 9.

### Validity-Scoring

In certain embodiments, the enriched health information dataset 900 is ranked for validity by source that may contain direct and/or indirect information, whereby a direct information may comprise information that is obtained directly from the patient, e.g., a laboratory result of a blood test, and indirect information may comprise a statement or record of others about the patient, such as, e.g., a medical record of a previous hospital stay. As explained above, direct information may be information that can be extracted directly from the EHRs, whereas indirect information may be data that can be assumed via the causative algorithms of embodiments of the invention. For example, if “Kleine-Levin Syndrome” is present as a documented diagnosis, it is possible to infer that “hypersomnia” was present as a symptom, because this syndrome is defined by recurrent hypersomnia and thus 100% of diagnosed patients should have this symptom even when not documented.

An exemplary ranking order includes:
1. Documented lab values/signs/biosignals
2. Medical classification codes (e.g., ICD) for diagnosis
3. Prescribed drugs
4. Symptoms described in free text

An enriched validity-scored health information dataset 314 may be generated which represents the actual phenotype of the patient. In certain embodiments, the enriched validity-scored health information dataset 314 combines at least part of the direct data 902 and indirect data 904 and the datapoints are scored for validity.

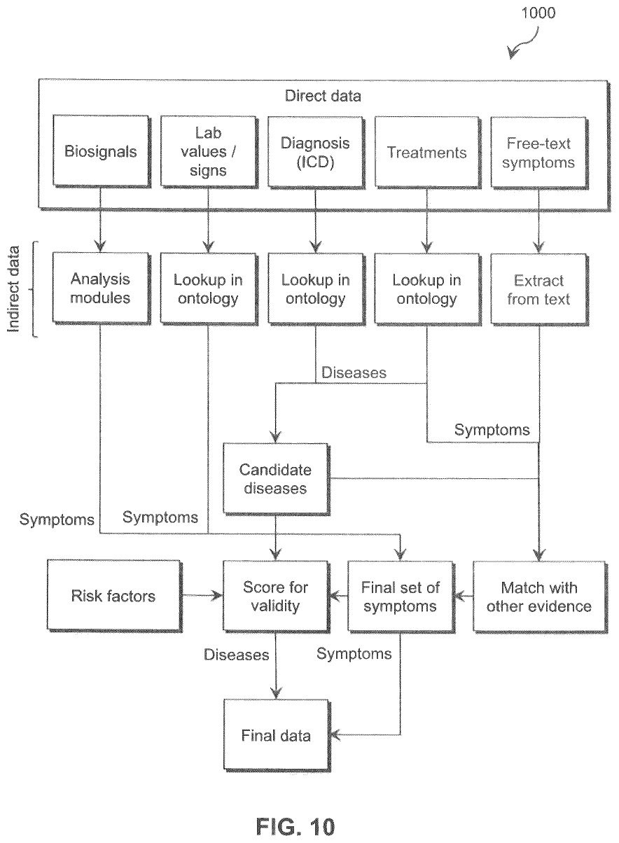

In the following, an embodiment of a validity-scoring process 1000 will be described with reference to FIG. 10.

### Lab Values/Signs/Biosignals:

Documented lab values/signs are looked up in the ontology 500 by their standard coding system (e.g., Logical Observation Identifiers Names and Codes; LOINC) and added as symptoms 506 to the final dataset 314 with a validity of 100%.

Biosignals, such as electrocardiogram (ECG) values are evaluated by specific analysis modules which map their results directly to the ontology 500. The assumed validity of the results of the analysis modules depends on the validation performance of each analysis module. As an example, consider an ECG with atrial fibrillation and an analysis module which is configured to detect that this is present in the ECG. For embodiments of the invention, it is less relevant how detecting atrial fibrillation in an ECG exactly works, but rather that “atrial fibrillation” as the output of an analysis module is the input which may then be used in embodiments of the invention. The results of the analysis modules are further validated by the accompanying written free-text clinical findings (see below).

### Medical Classification Codes for Diagnosis:

Diseases 504 matching the ICD code 502 for diagnosis are looked up in the ontology 500. Each resulting disease 504 is considered a candidate disease 504 and subsequently scored for validity according to the process description “Scoring diseases for validity” below.

### Treatments:

Symptoms 506 and diseases 504 for which the prescribed treatments, such as drugs 508, are indicated or commonly prescribed, even when wrongfully, are looked up in the ontology 500. Symptoms 506 that occur in any candidate disease 504 are added to the final data set 314 with an initial validity, which may, in one embodiment, be selected from the range of 40 to 80%, more preferably from the range of 50 to 70%, and which may in a practical example be approx. 60%.

Diseases 504 for which the prescribed drugs 508 are indicated or commonly prescribed, even when wrongfully, are added to the list of candidate diseases 504 and scored for validity according to the process description “Scoring diseases for validity” below.

### Symptoms in Free Text:

Symptoms 506 that are described in free text and are occurring in any disease 504 in the candidate disease list are added to the final data set 314 with an initial validity, which may, in one embodiment, be selected from the range of 60 to 100%, more preferably from the range of 70 to 90%, and which may in a practical example be approx. 80%.

If symptoms 506 are already present in the final data set 314 when added, the validity is boosted by a predefined value, e.g., by 20% (up to 100%).

Any symptom 506 that is described in the free text but not matching any candidate disease 504 is added to the final data set 314 with a validity of a predefined value, which may, in one embodiment, be selected from the range of 20 to 60%, more preferably from the range of 30 to 50%, and which may in a practical example be approx. 40%.

### Scoring Diseases for Validity:

Candidate diseases 502 resulting from the ontology lookup by diagnosis or drug indication are scored for validity by using risk factors such as the age and sex of the patient and all gathered symptoms with a validity >0 to get a list of possible causes. Scoring for validity is in one embodiment done by using an external differential diagnosis engine that returns as output a list of possible causes sorted by probability after risk factors and symptoms with validity >0 were given as input. Any candidate disease 502 that is in the list of possible causes will get a validity assigned based on the position (starting with 1) in the sorted list of possible causes according to the following formula: validity=(1/position)*100%

Finally, diseases 504 with a validity >0 are added to the final data set 314.

The result of the algorithm of the illustrated embodiment is an enriched validity-scored health information dataset 314 for each cluster 402 in the EHRs of a patient.

### Further Use of the Enriched Data

In certain embodiments, the enriched validity-scored health information dataset 314 comprises enhanced data points with missing data restored and/or a score for validity, which may be used for weighing parameters in further analysis steps.

For example, the output dataset 314 may be presented on an electronic display, so that a healthcare professional, such as a doctor, can make an improved diagnosis. Accordingly, embodiments of the invention can be used in computer-aided diagnostic. This way, the healthcare professional may be provided with a particularly complete and coherent data set to base his/her decision(s) on. False data may be filtered out and/or relevant information may be associated with specific diseases, which may be ranked by probability, as explained herein.

As another example, embodiments of the invention may also enable a greater accuracy for predictive machine learning models. Accordingly, the enriched data may be usable and/or used for optimizing a machine-learning model. In that case, the data may serve as training, validation and/or test data for the machine-learning model.

Yet another example is that of triage. Based on the information in the EHRs and/or based on the enriched data, patients may be prioritized as to the urgency of the required treatments, e.g., in an emergency room and/or certain treatments (e.g., an X-ray examination) may be performed before the first contact with a doctor and/or a suitable sequence of such treatments may be generated to employ the resources efficiently and timely.

Yet another example is to use the enriched data to recommend one or more actions to a patient, e.g., using an automated communication system such as a chat bot. Recommended actions may include a recommendation to wait and observe, a recommendation to take one or more generally available medications, a recommendation to make an appointment with a general health practitioner, a recommendation to make an appointment with a medical specialist, a recommendation to go to an emergency department immediately, or the like. It is even possible to automatically alter an emergency doctor or ambulance.

Although some aspects have been described in the context of an apparatus, it is clear that these aspects also represent a description of the corresponding method, where a block or device corresponds to a method step or a feature of a method step. Analogousty, aspects described in the context of a method step also represent a description of a corresponding block, or item, or feature, of a corresponding apparatus.

Some or all of the method steps may be executed by (or using) a hardware apparatus, such as a processor, a microprocessor, a programmable computer or an electronic circuit. Depending on certain implementation requirements, embodiments of the invention can be implemented in hardware or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments of the invention provide a data carrier having electronically readable control signals, which are capable of cooperating with a programmable computer system, such that one of the methods described herein is performed.

Generally, embodiments of the invention can be implemented as a computer program (product) with a program code, the program code being operative for performing one of the methods when the computer program product runs on a computer. The program code may, for example, be stored on a machine-readable carrier. Other embodiments comprise the computer program for performing one of the methods described herein, stored on a machine-readable carrier. In other words, an embodiment of the present invention is, therefore, a computer program having a program code for performing one of the methods described herein, when the computer program runs on a computer.

A further embodiment of the invention provides a storage medium (or a data carrier, or a computer-readable medium) comprising, stored thereon, the computer program for performing one of the methods described herein when it is performed by a processor. The data carrier, the digital storage medium or the recorded medium are typically tangible and/or non-transitionary. A further embodiment of the present invention is an apparatus as described herein comprising a processor and the storage medium.

A further embodiment of the invention provides a data stream or a sequence of signals representing the computer program for performing one of the methods described herein. The data stream or the sequence of signals may, for example, be configured to be transferred via a data communication connection, for example, via the Internet.

A further embodiment of the invention provides a processing means, for example, a computer or a programmable logic device, configured to, or adapted to, perform one of the methods described herein.

A further embodiment of the invention provides a computer having installed thereon the computer program for performing one of the methods described herein.

A further embodiment of the invention provides an apparatus or a system configured to transfer (e.g., electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

Certain embodiments of the invention may be based on using a machine-learning model or machine-learning algorithm. Machine learning may refer to algorithms and statistical models that computer systems may use to perform a specific task without using explicit instructions, instead relying on models and inference. For example, in machine-learning, instead of a rule-based transformation of data, a transformation of data may be used that is inferred from an analysis of historical and/or training data. For example, the content of images may be analyzed using a machine-learning model or using a machine-learning algorithm. In order for the machine-learning model to analyze the content of an image, the machine-learning model may be trained using training images as input and training content information as output. By training the machine-learning model with a large number of training images and/or training sequences (e.g., words or sentences) and associated training content information (e.g., labels or annotations), the machine-learning model “learns” to recognize the content of the images, so the content of images that are not included in the training data can be recognized using the machine-learning model. The same principle may be used for other kinds of sensor data as well: By training a machine-learning model using training sensor data and a desired output, the machine-learning model “learns” a transformation between the sensor data and the output, which can be used to provide an output based on non-training sensor data provided to the machine-learning model. The provided data (e.g., sensor data, meta data and/or image data) may be preprocessed to obtain a feature vector, which is used as input to the machine-learning model.

Machine-learning models may be trained using training input data. The examples specified above use a training method called “supervised learning”. In supervised learning, the machine-learning model is trained using a plurality of training samples, wherein each sample may comprise a plurality of input data values, and a plurality of desired output values, i.e., each training sample is associated with a desired output value. By specifying both training samples and desired output values, the machine-learning model “learns” which output value to provide based on an input sample that is similar to the samples provided during the training. Apart from supervised learning, semi-supervised learning may be used. In semi-supervised learning, some of the training samples lack a corresponding desired output value. Supervised learning may be based on a supervised learning algorithm (e.g., a classification algorithm, a regression algorithm or a similarity learning algorithm). Classification algorithms may be used when the outputs are restricted to a limited set of values (categorical variables), i.e., the input is classified to one of the limited set of values. Regression algorithms may be used when the outputs may have any numerical value (within a range). Similarity learning algorithms may be similar to both classification and regression algorithms but are based on learning from examples using a similarity function that measures how similar or related two objects are. Apart from supervised or semi-supervised learning, unsupervised learning may be used to train the machine-learning model. In unsupervised learning, (only) input data might be supplied and an unsupervised learning algorithm may be used to find structure in the input data (e.g. by grouping or clustering the input data, finding commonalities in the data). Clustering is the assignment of input data comprising a plurality of input values into subsets (clusters) so that input values within the same cluster are similar according to one or more (pre-defined) similarity criteria, while being dissimilar to input values that are included in other clusters. Reinforcement learning is a third group of machine-learning algorithms that may be used to train the machine-learning model. In reinforcement learning, one or more software actors (called “software agents”) are trained to take actions in an environment. Based on the taken actions, a reward is calculated. Reinforcement learning is based on training the one or more software agents to choose the actions such, that the cumulative reward is increased, leading to software agents that become better at the task they are given (as evidenced by increasing rewards).

Furthermore, some techniques may be applied to some of the machine-learning algorithms. For example, feature learning may be used. In other words, the machine-learning model may at least partially be trained using feature learning, and/or the machine-learning algorithm may comprise a feature learning component. Feature learning algorithms, which may be called representation learning algorithms, may preserve the information in their input but also transform it in a way that makes it useful, often as a pre-processing step before performing classification or predictions. Feature learning may be based on principal components analysis or cluster analysis, for example.

In some examples, anomaly detection (i.e., outlier detection) may be used, which is aimed at providing an identification of input values that raise suspicions by differing significantly from the majority of input or training data. In other words, the machine-learning model may at least partially be trained using anomaly detection, and/or the machine-learning algorithm may comprise an anomaly detection component.

In some examples, the machine-learning algorithm may use a decision tree as a predictive model. In other words, the machine-learning model may be based on a decision tree. In a decision tree, observations about an item (e.g., a set of input values) may be represented by the branches of the decision tree, and an output value corresponding to the item may be represented by the leaves of the decision tree. Decision trees may support both discrete values and continuous values as output values. If discrete values are used, the decision tree may be denoted a classification tree, if continuous values are used, the decision tree may be denoted a regression tree.

Association rules are a further technique that may be used in machine-learning algorithms. In other words, the machine-learning model may be based on one or more association rules. Association rules are created by identifying relationships between variables in large amounts of data. The machine-learning algorithm may identify and/or utilize one or more relational rules that represent the knowledge that is derived from the data. The rules may e.g. be used to store, manipulate or apply the knowledge.

Machine-learning algorithms are usually based on a machine-learning model. In other words, the term “machine-learning algorithm” may denote a set of instructions that may be used to create, train or use a machine-learning model. The term “machine-learning model” may denote a data structure and/or set of rules that represents the learned knowledge (e.g., based on the training performed by the machine-learning algorithm). In embodiments, the usage of a machine-learning algorithm may imply the usage of an underlying machine-learning model (or of a plurality of underlying machine-learning models). The usage of a machine-learning model may imply that the machine-learning model and/or the data structure/set of rules that is the machine-learning model is trained by a machine-learning algorithm.

For example, the machine-learning model may be an artificial neural network (ANN). ANNs are systems that are inspired by biological neural networks, such as can be found in a retina or a brain. ANNs comprise a plurality of interconnected nodes and a plurality of connections, so-called edges, between the nodes. There are usually three types of nodes, input nodes that receive input values, hidden nodes that are (only) connected to other nodes, and output nodes that provide output values. Each node may represent an artificial neuron. Each edge may transmit information, from one node to another. The output of a node may be defined as a (non-linear) function of its inputs (e.g., of the sum of its inputs). The inputs of a node may be used in the function based on a “weight” of the edge or of the node that provides the input. The weight of nodes and/or of edges may be adjusted in the learning process. In other words, the training of an artificial neural network may comprise adjusting the weights of the nodes and/or edges of the artificial neural network, i.e. to achieve a desired output for a given input.

Alternatively, the machine-learning model may be a support vector machine, a random forest model or a gradient boosting model. Support vector machines (i.e., support vector networks) are supervised learning models with associated learning algorithms that may be used to analyze data (e.g., in classification or regression analysis). Support vector machines may be trained by providing an input with a plurality of training input values that belong to one of two categories. The support vector machine may be trained to assign a new input value to one of the two categories. Alternatively, the machine-learning model may be a Bayesian network, which is a probabilistic directed acyclic graphical model. A Bayesian network may represent a set of random variables and their conditional dependencies using a directed acyclic graph. Alternatively, the machine-learning model may be based on a genetic algorithm, which is a search algorithm and heuristic technique that mimics the process of natural selection.

1. A computer-implemented method for enriching ambiguous, incomplete or sparse health data, comprising the steps of:
obtaining an input dataset comprising a plurality of electronic health records associated with a patient;
extracting health information that is explicitly recited in the electronic health records from the input dataset, including at least one diagnosis indicated by a name of a disease or a medical classification code which denotes the disease;
generating supplementary health information that is not explicitly documented in the electronic health records based, at least in part, on the extracted health information, the supplementary health information including at least one or more symptoms inferred from the disease directly or indirectly documented in the input dataset; and
validity-scoring at least part of the extracted health information and the supplementary health information to produce an output dataset.

2. The method of claim 1, wherein of generating the supplementary health information comprises determining, using a code-disease mapping one or more diseases associated with the medical classification code documented in the input dataset.

3. The method of claim 1, wherein of generating the supplementary health information comprises determining, using a disease-symptom mapping, the at least one or more symptoms associated with the disease documented in the input dataset and/or determined using a code-disease mapping.

4. The method of claim 1, wherein the generating the supplementary health information comprises determining, using a drug-symptom mapping and/or a drug-disease mapping, one or more symptoms and/or diseases associated with a drug documented in the input dataset.

5. The method of claim 1, wherein the generating the supplementary health information is based on an ontology;
wherein, the ontology comprises the code-disease mapping, the disease-symptom mapping, the drug-symptom mapping and/or the drug-disease mapping.

6. The method of claim 1, wherein the validity-scoring comprises ranking diseases and/or symptoms based on a credibility associated with a source of a respective disease and/or a symptom;
wherein documented lab values, signs and/or biosignals indicate a highest credibility;
wherein medical classification codes used for a diagnosis indicate a second highest credibility;
wherein prescribed treatments and/or drugs indicate a third highest credibility; and
wherein symptoms documented in free text indicate a lowest credibility.

7. The method of claim 1, wherein the validity-scoring comprises one or more of the following:
scoring a symptom derived from a lab value or a sign with a first validity factor, wherein the first validity factor is preferably 100%;
scoring a symptom derived from a biosignal with a second validity factor, wherein the second validity factor depends on an analysis module associated with the biosignal;
scoring a disease derived from a diagnosis or a prescribed treatment with a third validity factor, wherein the third validity factor is based, at least in part, on one or more risk factors of the patient, if present in the input dataset.

8. The method of claim 1, wherein each of a plurality of electronic health records comprises a timestamp and wherein the method further comprises:
sorting the input dataset by the timestamp; and
clustering the input dataset into one or more clusters based, at least in part, on the timestamp;
wherein the extracting health information is performed for each cluster.

9. The method of claim 8, wherein obtaining the input dataset comprises:
exporting the plurality of electronic health records from a hospital information system, wherein the exported plurality of electronic health records comprises all electronic health records associated with the patient available in the hospital information system; and
anonymizing the exported plurality of electronic health records;
wherein exporting and anonymizing is performed by a data processing system that is deployed locally within an Information Technology infrastructure of a hospital comprising the hospital information system, wherein, the data processing system is for communicating with the hospital information system only via a secured local network connection.

10. The method of, wherein the step of extracting health information comprises processing the input dataset using a feature extraction method for text classification.

11. The method of claim 1, further comprising outputting the output dataset on a display of an electronic device;
wherein, the electronic device is associated with a healthcare professional for use in computer-aided diagnosis; or the electronic device is associated with the patient.

12. The method of claim 1, further comprising providing the output dataset as an input to a computer system for further use, and/or to a machine-learning model.

13. The method of claim 1, further comprising, based at least on the enriched health data, prioritizing patients as to the urgency of required treatments, in an emergency room.

14. The method of claim 1, further comprising, based at least on the enriched health data, causing performance of certain treatments, such as an X-ray examination, before the first contact with a doctor.

15. The method of claim 1, further comprising, based at least on the enriched health data, generating a sequence of treatments to be performed.

16. The method of claim 1, further comprising, based at least on the enriched health data, recommending one or more actions to the patient using an automated communication system such as a chat bot.

17. A computer program comprising instructions which, when the program is executed by a computer, cause the computer to carry out the method of claim 1.

18. A data processing system comprising means for carrying out the method of claim 1.

19. The data processing system of claim 18, being deployed locally within an Information Technology infrastructure of a hospital comprising a hospital information system, wherein, the data processing system is for communicating with the hospital information system only via a secured local network connection.
